(19) United States
(12) Patent Application Publication
(10) Pub. No.: US 2024/0261351 A1
(43) Pub. Date: Aug. 8, 2024

(54) COMPOSITIONS AND METHODS FOR THE TREATMENT OF BONE AND JOINT DISEASES

(71) Applicant: Horace Osborne FERGUSON, Ocala, FL (US)

(72) Inventor: Horace Osborne FERGUSON, Ocala, FL (US)

(21) Appl. No.: 18/638,326

(22) Filed: Apr. 17, 2024

Related U.S. Application Data
(62) Division of application No. 17/969,941, filed on Oct. 20, 2022.
(60) Provisional application No. 63/360,687, filed on Oct. 21, 2021.

(51) Int. Cl. A61K 36/04; A61K 33/10; A61K 45/06; A61P 19/08

(52) U.S. Cl. CPC A61K 36/04; A61K 33/10; A61K 45/06; A61P 19/08

(57) ABSTRACT

A novel composition for the treatment of developmental bone diseases, and other related ailments in a subject using a composition comprising an algae extract and at least one strontium salt is provided. A treatment of developmental bone diseases and other related ailments in a subject by administering a composition comprising a non-viable algal culture or an extract thereof and a second composition comprising at least one strontium salt is also provided. The algae extract can comprise calcium carbonate and magnesium carbonate.

### CROSS-REFERENCE TO RELATED APPLICATIONS

The present application is a divisional of U.S. patent application Ser. No. 17/969,941, filed Oct. 20, 2022, which claims the benefit of U.S. Patent Application Ser. No. 63/360,687, filed Oct. 21, 2021, each of which is hereby incorporated by reference in its entirety, including any tables, figures, or drawings.

### BACKGROUND OF THE INVENTION

The thoroughbred horse racing industry has long been plagued by lameness, affecting about a third of all horses. Horses and other animals, including pigs, dogs, cats, chickens, mules, donkeys, cattle, sheep, goats, llamas, and human children, can become lame during rapid growth or intense training, as a variety of degenerative diseases can cause orthopedic pain in the musculoskeletal system. In the case of horses, lameness restricts the overall movement and athletic performance of racehorses, which costs the industry over a billion dollars annually in lost use of the racehorses and in medical treatment.

Osteochondritis dissecans (OCD) is a significant disease damaging the bone and cartilage of joints, primarily in young and fast-growing humans and animals, which can induce lameness. The most common locations affected by OCD include, for example, the elbow, knee, ankle, stifle, hock, and fetlock joints. OCD occurs when an abnormal blood supply fails to calcify bone, causing the bone to become fragile and break loose from its attaching cartilage. This condition disrupts the skeletal structure of the joint, which can induce pain and inhibit movement, resulting in an OCD defect on the bone surface and OCD fragments in the joint. Furthermore, the causes of improper calcification are largely unknown, making treatment methods hard to develop.

OCD and similar diseases in humans and animals can result in bone cysts. These are cavities in a region filled with fluid that has not grown into bone. While some bone cysts are benign and naturally heal over time with fibrous tissue, the condition weakens the bone structure and increases its fragility. Other bone cysts may be persistent with the potential to enlarge. For example, active bone cysts develop next to growth plates, potentially expanding and further damaging the bone structure. Consequently, racehorses with bone cysts must limit their training to avoid bone fractures.

Osteoarthritis is another major factor in lameness, as it can generate joint pain and stiffness in humans and animals. Generally, these diseases cause joint inflammation and deformity, especially to racehorses, which can deteriorate bone (i.e., lysis) or form osteophytes (i.e., spurs). Osteoarthritis also increases the potential of re-injury, as it can cause deterioration or form osteophytes in key joints, including, for example, knees and ankles (e.g., supracondylar lysis).

Diseases that cause bone inflammation, such as sesamoiditis, are another significant problem for racehorses. Due to the sesamoid bone's proximity to the fetlock joint in horses, the excessive pressures created during training often cause the sesamoid bones and surrounding tendons to become inflamed. The stress can also tear adjacent vascular channels and suspensory branches, often resulting in ligament injuries. In some cases, the bone may even demineralize, weakening to the point of fracturing.

Other traumatic bone injuries caused by diseases can also occur. Lack of bone calcification in the fetlock region can cause bone bruising, possibly through bone compression and a loss of blood supply. Sclerosis occurs when stress causes bone to harden and decrease flexibility, which may be detrimental to the racehorse's performance. A similar process also causes navicular bone deformation. As the tissues in the navicular region of the forefoot become inflamed, the bone conforms abnormally, resulting in growth defects.

The immense complications associated with these problems, accompanied by the potential to discover suitable treatment methods for corresponding human conditions, have prompted significant attention from researchers to study these diseases. However, most research efforts emphasize developing methods for diagnosing such diseases.

Some of the available treatment methods for OCD involve surgical procedures or devices that tend to be expensive. More cost-effective treatments include those providing food supplements in animal feed. However, these methods generally aim to treat the symptoms of OCD, as opposed to addressing the original problem of improper bone calcification. For example, specific therapeutic compounds can cushion the joint by creating a more stabilized structure. While this treatment helps alleviate the symptoms of pain and swelling, it does not help to completely restore the fragmented bone or OCD defects.

Bone cyst medical treatments have encountered similar problems, as there are very few proposed treatment methods to effectively cure the condition. Current treatments include administering hormones that promote bone growth, including the parathyroid hormone. However, excessive parathyroid hormone levels cause hyperparathyroidism, which can result in shifting lameness from one leg to another. Thus, this treatment is an infeasible approach to treating bone cysts in horses.

Most methods and solutions for treating osteoarthritis in humans and animals can only offer to prevent or reduce the symptoms. As a result, osteoarthritis is does not have treatment methods to repair the damaged cartilage and bone.

Despite the significance of sesamoiditis in the horse racing industry, there remain few treatment routes. Similarly, other horse-specific injuries, including navicular bone deformation, do not have consistent treatments. Furthermore, these treatments generally offer symptomatic relief rather than providing a complete cure for the condition.

Given the impact of these bone and joint diseases on the horse racing industry, treatment methods that only alleviate the symptoms are not enough. In addition, general cures for the wide variety of pain-inducing diseases and injuries are yet unknown. Therefore, there remains a need for compositions and methods of treatment that treat the systemic problems in these diseases and ailments.

### BRIEF SUMMARY OF THE INVENTION

The subject invention relates to a novel composition for the treatment for bone and joint diseases and other related ailments in a subject by administering a composition comprising an non-viable algal culture or an extract thereof and at least one strontium salt. The subject invention also relates to two compositions for the treatment of developmental bone diseases and other related ailments in a subject by administering a composition comprising an non-viable algal culture or an extract thereof and a second composition comprising at least one strontium salt. In certain embodiments, the methods can inhibit the progress of the developmental bone disease.

In certain embodiments, one or more algae species can be used, including, for example, Lithothamnion spp. The non-viable algal culture or extract thereof can comprise calcium carbonate and magnesium carbonate with various trace minerals and vitamins.

In certain embodiments, the developmental bone disease is osteochondrosis (OCD), sesamoiditis, bone sclerosis, osteoarthritis, navicular disease, or any combination thereof. In certain embodiments, the related ailment is bone bruising, cartilage damage, bone fracture, or post-infection osteomyelitis bone damage.

### BRIEF DESCRIPTION OF THE DRAWINGS

FIG. 1. Resolution of Sesamoiditis in horses after treatment with a first composition comprising a marine mineral complex and a second composition comprising a strontium salt.

FIG. 2. Resolution of OCD Stifle LTR/MTR Defects in horses after treatment with a first composition comprising a marine mineral complex and a second composition comprising a strontium salt.

FIG. 3. Resolution of OCD Stifle medial femoral condyle (MFC) cysts in horses after treatment with a first composition comprising a marine mineral complex and a second composition comprising a strontium salt.

FIG. 4. Resolution of OCD metacarpal III/first phalanx bone P-I (MCIII/PI) cysts in horses after treatment with a first composition comprising a marine mineral complex and a second composition comprising a strontium salt.

FIG. 5. Resolution of navicular disease in horses after treatment with a first composition comprising a marine mineral complex and a second composition comprising a strontium salt.

FIG. 6. OCD Cyst 1 Right Stifle Caudocranial view on day 72

FIG. 7. OCD Cyst 1 Right Stifle Oblique view on day 72

FIG. 8. OCD Cyst 1 Right Stifle Caudocranial view on day 106

FIG. 9. OCD Cyst 1 Right Stifle Oblique view on day 106

FIG. 10. OCD Cyst 1 Right Stifle Caudocranial view on day 1

FIG. 11. OCD Cyst 1 Right Stifle Oblique view on day 1

FIG. 12. OCD Cyst 1 Right Stifle Oblique view on day 34

FIG. 13. OCD Cyst 2 Left Fore Fetlock Dorsopalmar view on day 75

FIG. 14. OCD Cyst 2 Left Hind Fetlock Dorsopalmar view on day 75

FIG. 15. OCD Cyst 2 Left Fore Fetlock Dorsopalmar view on day 118

FIG. 16. OCD Cyst 2 Left Hind Fetlock Dorsopalmar view on day 118

FIG. 17. OCD Cyst 2 Left Fore Fetlock Dorsopalmar view on day 1

FIG. 18. OCD Cyst 2 Left Hind Fetlock Dorsopalmar view on day 1

FIG. 19. OCD Cyst 2 Left Fore Fetlock Dorsopalmar view on day 33

FIG. 20. OCD Defect 1 Left Stifle Oblique view on day 82

FIG. 21. OCD Defect 1 Left Stifle Lateral view on day 118

FIG. 22. OCD Defect 1 Left Stifle Oblique view on day 118

FIG. 23. OCD Defect 1 Left Stifle Lateral view on day 7

FIG. 24. OCD Defect 1 Left Stifle Oblique view on day 7

FIG. 25. OCD Defect 1 Left Stifle Oblique view on day 42

FIG. 26. OCD Defect 2 Left Stifle Oblique view on day 47

FIG. 27. OCD Defect 2 Left Stifle Oblique view on day 83

FIG. 28. OCD Defect 2 Left Stifle Lateral view on day 125

FIG. 29. OCD Defect 2 Left Stifle Oblique view on day 125

FIG. 30. OCD Defect 2 Left Stifle Lateral view on day 163

FIG. 31. OCD Defect 2 Left Stifle Oblique view on day 163

FIG. 32. OCD Defect 2 Left Stifle Lateral view on day 224

FIG. 33. OCD Defect 2 Left Stifle Oblique view on day 224

FIG. 34. OCD Defect 2 Left Stifle Lateral view on day 12

FIG. 35. OCD Defect 2 Left Stifle Oblique view on day 12

FIG. 36. Sesamoiditis 1 Left Fore Fetlock Dorsolateral-Palmaromedial Oblique View on day 134

FIG. 37. Sesamoiditis 1 Right Fore Fetlock Dorsolateral-Palmaromedial Oblique View on day 134

FIG. 38. Sesamoiditis 1 Left Fore Fetlock Dorsolateral-Palmaromedial Oblique View on day 189 FIG. 39. Sesamoiditis 1 Right Fore Fetlock Dorsolateral-Palmaromedial Oblique View on day 189

FIG. 40. Sesamoiditis 1 Left Fore Fetlock Dorsolateral-Palmaromedial Oblique View on day 226

FIG. 41. Sesamoiditis 1 Right Fore Fetlock Dorsolateral-Palmaromedial Oblique View on day 226

FIG. 42. Sesamoiditis 1 Left Fore Fetlock Dorsolateral-Palmaromedial Oblique View on day 254

FIG. 43. Sesamoiditis 1 Right Fore Fetlock Dorsolateral-Palmaromedial Oblique View on day 254

FIG. 44. Sesamoiditis 1 Right Fore Fetlock Dorsolateral-Palmaromedial Oblique View on day 285

FIG. 45. Sesamoiditis 1 Left Fore Fetlock Dorsolateral-Palmaromedial Oblique View on day 285

FIG. 46. Sesamoiditis 2 Left Fore Fetlock Dorsomedial-Palmarolateral Oblique View on day 55

FIG. 47. Sesamoiditis 2 Sonogram of Left Fore Fetlock Dorsomedial-Palmarolateral Oblique View on day 55 FIG. 48. Sesamoiditis 2 Left Fore Fetlock Dorsomedial-Palmarolateral Oblique View on day 91

FIG. 49. Sesamoiditis 2 Left Fore Fetlock Dorsomedial-Palmarolateral Oblique View on day 132

FIG. 50. Sesamoiditis 2 Sonogram of Left Fore Fetlock Dorsomedial-Palmarolateral Oblique View on day 132

FIG. 51. Sesamoiditis 2 Left Fore Fetlock Dorsomedial-Palmarolateral Oblique View on day 189

FIG. 52. Sesamoiditis 2 Left Fore Fetlock Dorsomedial-Palmarolateral Oblique View on day 226

FIG. 53. Sesamoiditis 3 Left Fore Fetlock Dorsolateral-Palmaromedial Oblique View on day 76 FIG. 54. Sesamoiditis 3 Left Fore Fetlock Dorsolateral-Palmaromedial Oblique View on day 104

FIG. 55. Sesamoiditis 3 Sonogram of Left Fore Fetlock Dorsolateral-Palmaromedial Oblique View on day 104

FIG. 56. Sesamoiditis 3 Left Fore Fetlock Dorsolateral-Palmaromedial Oblique View on day 39

FIG. 57. Sesamoiditis 3 Left Fore Fetlock Dorsolateral-Palmaromedial Oblique View on day 10

FIG. 58. Sesamoiditis 3 Sonogram of Left Fore Fetlock Dorsolateral-Palmaromedial Oblique View on day 10

FIG. 59. Sesamoiditis 3 Left Fore Fetlock Dorsolateral-Palmaromedial Oblique View on day 40

### DETAILED DISCLOSURE OF THE INVENTION

The subject invention relates to a novel composition for the treatment for bone and joint diseases and other related ailments in a subject by administering a composition comprising an non-viable algal culture or an extract thereof and at least one strontium salt. The subject invention also relates to two compositions for the treatment for OCD, osteoarthritis, sesamoiditis, navicular disease, and other related diseases in a subject using a first composition comprising an algae extract and a second composition comprising at least one strontium salt.

### Definitions

Ranges provided herein are understood to be shorthand for all of the values within the range. For example, a range of 1 to 20 is understood to include any number, combination of numbers, or sub-range from the group consisting 1, 2, 3, 4, 5, 6, 7, 8, 9, 10, 11, 12, 13, 14, 15, 16, 17, 18, 19 and 20, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein a “reduction” means a negative alteration, and an “increase” means a positive alteration, wherein the negative or positive alteration is at least 0.001%, 0.01%, 0.1%, 0.5%, 1%, 5%, 10%, 15%, 20%, 25%, 30%, 35%, 40%, 45%, 50%, 55%, 60%, 65%, 70%, 75%, 80%, 85%, 90%, 95% or 100%. The term “modulate” is meant to alter (increase or decrease). Such alterations are detected by standard methods known in the art such as those described herein.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially of” the recited component(s).

Unless specifically stated or obvious from context, as used herein, the term “or” is understood to be inclusive. Unless specifically stated or obvious from context, as used herein, the terms “a,” “and” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used herein, “algae” generally refers to microscopic or macroscopic eukaryotic photosynthetic organisms. As used herein, the term “marine algae” may describe without limitation one or more (i.e., one or a combination) brown algae, green algae, or red algae. The marine algae can be, for example, any of the following red algae genera: Lithothamnion, Leptophytum, Melobesioidea, Mesophyllum, or Phymatolithon. The compounds of the present disclosure will be useful in treating one or more developmental bone disease, particularly but not necessarily exclusively involving horses.

As used herein, the term “pharmaceutically acceptable” means compatible with the other ingredients of a pharmaceutical composition and not deleterious to the recipient thereof.

As used herein, the term “subject” refers to a animal. Examples of subjects include mammals, such as, for example, humans, dogs, cows, horses, pigs, sheep, goats, cats, mice, rabbits, rats, and transgenic non-human animals. In certain embodiments, the subject is a horse, such as, for example, a horse suffering from, at risk of suffering from, or susceptible to a developmental bone disease.

As used herein, reference to a “algal-based composition” or “algal-sourced composition” means a composition that comprises components that were produced as the result of the growth of algae or other cell cultures. A algal-based composition may comprise the algae cells themselves, or the algae may be separated from the broth, media, or seawater in which they were grown, and the composition comprises residual cellular components, depositions, and/or by-products of algal growth. Preferably, the compositions according to the subject invention have been derived from non-viable algae. The by-products of algal growth may be, for example, metabolites, cell membrane components, synthesized proteins, depositions on the algae (e.g., calcium carbonate and magnesium carbonate) and/or other cellular components.

The subject invention further provides “algal-based products,” which are products that are to be applied in practice to achieve a desired result. The algal-based product may comprise further ingredients that have been added. These additional ingredients can include, for example, strontium salts, stabilizers, buffers, and/or appropriate carriers (e.g., water or salt solutions). The algal-based product may comprise mixtures of algal-based compositions. The algal-based product may also comprise one or more components of a algal-based composition that have been processed in some way such as, but not limited to, filtering, centrifugation, lysing, drying, purification, and the like.

As used herein, “harvested” refers to removing some or all of the algal-based composition from a growth vessel or from a naturally-occurring source.

For the purposes of this invention the terms “treatment, treating, treat” or equivalents of these terms refer to healing, alleviating, relieving, altering, remedying, ameliorating, improving, or affecting the condition or the symptoms of a subject suffering with a disease or condition, for example, a developmental bone disease or related ailment. The subject to be treated can be suffering from or at risk of developing the disorder or condition, for example, a developmental bone disease or related ailment. When provided therapeutically, the compound can be provided before the onset of a symptom. The therapeutic administration of the substance serves to attenuate any actual symptom.

For the purposes of this invention, the terms “preventing, preventive, prophylactic” or equivalents of these terms are indicate that the compounds of the subject invention are provided in advance of any disease symptoms and are a separate aspect of the invention (i.e., an aspect of the invention that is distinct from aspects related to the terms “treatment, treating, treat” or equivalents of these terms which refer to healing, alleviating, relieving, altering, remedying, ameliorating, improving, or affecting the condition or the symptoms of a subject suffering from a developmental bone disease or related ailment). The prophylactic administration of the compounds of the subject invention serves to prevent, reduce the likelihood, or attenuate one or more subsequent symptoms or condition.

By “therapeutically effective dose,” “therapeutically effective amount”, or “effective amount” is intended to be an amount of a compounds of the subject invention disclosed herein that, when administered to a subject, decreases the number or severity of symptoms or inhibits or eliminates the progression or initiation of a developmental bone disease or related ailment or reduces any increase in symptoms, or improves the clinical course of the disease or ailment as compared to untreated subjects. “Positive therapeutic response” refers to, for example, improving the condition of at least one of the symptoms of a developmental bone disease or related ailment.

An effective amount of the therapeutic agent is determined based on the intended goal. The term “unit dose” refers to a physically discrete unit suitable for use in a subject, each unit containing a predetermined quantity of the therapeutic composition calculated to produce the desired response in association with its administration, i.e., the appropriate route and treatment regimen. The quantity to be administered, both according to number of treatments and unit dose, depends on the subject to be treated, the state of the subject and the protection desired. Precise amounts of the therapeutic composition also depend on the judgment of the practitioner and are particular to each individual. Generally, the dosage of the compounds of the subject invention will vary depending upon such factors as the subject's age, weight, girth, height, sex, general medical condition and previous medical history.

In some embodiments of the invention, the method comprises administration of multiple doses of the compounds of the subject invention. The method may comprise administration of 1, 2, 3, 4, 5, 6, 7, 8, 9, 10, 15, 20, 25, 30, 35, 40, 50, 75, 100, 150, 200, 250, 300, 350, 400, 450, 500, 600, 700, 800, 900, 1000 or more therapeutically effective doses of a composition comprising the compounds of the subject invention as described herein. In some embodiments, doses are administered over the course of 1 day, 2 days, 3 days, 4 days, 5 days, 6 days, 7 days, 10 days, 14 days, 21 days, 30 days, 2 months, 3 months, 6 months, 1 year, 2 years, 3 years, 4 years, 5 years, 6 years, 7 years, 8 years, 9 years, 10 years, or more than 10 years. The frequency and duration of administration of multiple doses of the compositions is such as to inhibit or delay the initiation of a developmental bone disease or related ailment or reduce the symptoms of a developmental bone disease or related ailment. Moreover, treatment of a subject with a therapeutically effective amount of the compounds of the invention can include a single treatment or can include a series of treatments. It will also be appreciated that the effective dosage of a compound used for treatment may increase or decrease over the course of a particular treatment. Changes in dosage may result and become apparent from the results of diagnostic methods for detecting a developmental bone disease, related ailment, or symptoms thereof known in the art. In some embodiments of the invention, the method comprises administration of the compounds at a single time per day or several times per day, including but not limiting to 2 times per day, 3 times per day, and 4 times per day.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof, and from the claims. All references cited herein are hereby incorporated by reference.

### Compositions

This disclosure provides a novel composition for the treatment for OCD, osteoarthritis, sesamoiditis, navicular disease, and other diseases or ailments in a subject using a composition comprising an algae extract and at least one strontium salt. In preferred embodiments, two distinct compositions are used for the treatment of OCD, osteoarthritis, sesamoiditis, navicular disease, and other diseases or ailments in a subject in which a first composition comprises an algae extract (i.e., marine mineral complex) and a second composition comprises at least one strontium salt.

The compositions utilize a natural multi-mineral complex sourced from algae. In preferred embodiments, the algae are marine algae. In more preferred embodiments, the algae are red algae. In the most preferred embodiments, the extract may be produced from Lithothamnion spp., including, for example, Lithothamnion calcareum, Lithothamnion corallioides, and Lithothamnion crispatum. The algae used may be found in their natural state (e.g., naturally growing on the seafloor) or cultivated in an environment with sufficient nutrients. The algae can be free-living or attached to substrates, including rocks, coral, shells, and other organisms and algae. The mineral complex derives from calcium-secreting red algae, specifically their calcified skeleton. In certain embodiments, the extract is obtained from specific portions of the coralline algae, for example, the algae's branches or fronds. In other embodiments, particularly those using smaller species of calcified algae, the extract may be obtained from the entire organism.

In certain embodiments, the algae are ready to be harvested when algal growth is completed. In certain embodiments, this occurs when the calcified sections of the alga detach from the rest of the alga or when the entire alga detaches from its substrate. The algae are collected using standard harvesting techniques depending on where they accumulate. For example, detached algae that sink, such as, for example, to the ocean floor can be dredged up; algae that wash ashore can be gathered by hand; and algae grown in a bioreactor or other growth chamber or aquaculture can be harvested continually. Once the coralline algae are harvested, they can be filtered and purified using methods well-known in the art. These methods include, for example, chemical disinfection, such as, for example, with the use of bleaching agents, hydrogen peroxide, and/or antiseptic substances; heat disinfection, such as, for example, steam sterilization; electromagnetic radiation, such as, for example, ultraviolet light irradiation; high-pressure processing, such as, for example, pascalization; or any combinations thereof. In certain embodiments, the methods further comprise heating and/or desiccating the algal cells and milling, grinding, pulverizing, or any other method of reducing and/or homogenizing the size of the algae-based cultures or extracts thereof.

In preferred embodiments, the algae extract comprises Aquamin® (Marigot Ltd, Corra Binne, Ireland), a commercially available product that is made from harvested natural calcareous red algae growing off the coasts of Iceland and Ireland. In these conditions, the algae fronds absorb minerals from surrounding seawater into their calcareous cell walls. The resulting mineral components are about 85% calcium carbonate and 8.5% magnesium carbonate by weight, with trace minerals including, for example, aluminum, antimony, arsenic, barium, beryllium, bismuth, boron, bromine, cadmium, cerium, cesium, chloride, chromium, cobalt, copper, dysprosium, erbium, curopium, fluoride, gadolinium, gallium, germanium, gold, hafnium, holmium, indium, iodine, iridium, iron, lanthanum, lead, lithium, lutetium, manganese, mercury, molybdenum, neodymium, nickel, niobium, osmium, palladium, phosphorus, platinum, potassium, prascodymium, rhenium, rhodium, rubidium, ruthenium, samarium, scandium, selenium, silicon, silver, sodium, strontium, sulfur, tantalum, tellurium, terbium, thallium, thorium, thulium, tin, titanium, tungsten, vanadium, ytterbium, yttrium, zinc, and zirconium. When the algae fronds die, they break off and accumulate on the seabed, where they are harvested. Then, the calcified algae fronds are washed and screened before being treated with hydrogen peroxide for sterilization and deodorization. Afterward, the mineral complex is dried and milled using sterile equipment to obtain crude extracts.

The compositions further comprise any strontium salt or combination of strontium salts. Strontium is known to have the dual action function of increasing bone deposition (i.e., osteoblast activity) and inhibiting bone resorption (i.e., osteoclast activity). In combination with calcium, strontium also has been shown to potentially increase bone mineral density and decrease cartilage degradation.

In certain embodiments, the compositions comprise a strontium salt, such as, for example, strontium ranelate, strontium citrate, strontium phosphate, strontium succinate, strontium glutamate, strontium ketoglutarate, strontium maleate, strontium pyruvate, strontium threonate, strontium chloride, strontium chloride hexahydrate, strontium malonate, strontium fumarate, strontium ascorbate, strontium aspartate, strontium tartrate, strontium methanesulfonate, strontium benzenesulfonate, strontium bromide, strontium bromide hexahydrate, strontium acetate, strontium carbonate, strontium gluconate, strontium lactate, and any other strontium salts of an organic or inorganic acid. In certain embodiments, the compositions further comprise a mixture of two or more of any strontium salts, including those listed above.

In preferred embodiments, strontium ranelate is used in the subject compositions. In other preferred embodiments, strontium citrate or strontium phosphate may be used in the compositions instead of strontium ranelate.

In certain embodiments, the compositions further comprise vitamins, including, for example, vitamin A (retinoids and carotene), vitamin B₁ (thiamin), vitamin B₂ (riboflavin), vitamin B₃ (niacin), vitamin B₅ (pantothenic acid), vitamin B₆ (pyridoxine), vitamin B₁₂ (cobalamin), biotin, vitamin C (ascorbic acid), choline, vitamin D (calciferol), vitamin E (alpha-tocopherol), vitamin B₉ (folic acid), and vitamin K (phylloquinone, menadione).

If required, the components of the compositions (e.g., algae extract and strontium salts) may be further processed and supported with additives to be in the proper form for mixing and administering. Further processing may include grinding into a powder or dispersing in a solvent including, for example, water, ethanol, butanol, propanol, aliphatic hydrocarbons, alicyclic hydrocarbons, xylene, toluene, ketones, isopropyl alcohol, and mixtures thereof.

In preferred embodiments, the compositions are mixtures comprising the processed algae extract and strontium salts. In a preferred embodiment, the composition comprising the algae extract remains distinct from the strontium salt composition. The algae extract may be included in the compositions at concentrations from about 0.1 to about 100% by weight, preferably from about 5 to about 99% by weight, and more preferably from about 20 to about 90% by weight. The strontium salts may be included in the mixture at concentrations from about 0.1 to about 100% by weight, preferably from about 5 to about 99% by weight, and more preferably from about 20 to about 90% by weight. In other embodiments, the compositions include algae extract at concentrations from about 0.01 to about 50% by weight, preferably from about 0.1 to about 25% by weight, and more preferably from about 0.1 to about 10% by weight. The strontium salts may be included in the mixture at concentrations from about 0.01 to about 50% by weight, preferably from about 0.1 to about 25% by weight, and more preferably from about 0.1 to about 10% by weight. In certain embodiments, the ratio by weight between the algae extract and the strontium salts is from about 0.05 (1:20) to about 20 (20:1), preferably from about 0.1 (1:10) to about 10 (10:1), and more preferably from about 0.2 (1:5) to about 5 (5:1).

Additional components which may be added to enhance the compositions and their delivery include, for example, fillers, binders, disintegrants, diluents, glidants, emulsifying agents, suspending agents, stabilizers, enhancers, flavors, dyes, pigments, pH adjusting agents, buffers, retarding agents, wetting agents, adhesion-promoting compounds, surface active agents, preservatives, antioxidant, defoamers, anti-skinning agents, texturizers, emulsifying agents, lubricants, solubility controlling agents, chelating agents, conventional carrier compositions, and mixtures thereof.

The compositions can be administered for treatment through several delivery methods. Methods of oral drug delivery include, for example, tablets, capsules, sachets, powders, pellets, granules, granulates, mixtures, syrups, solutions, suspensions, or emulsions. Methods of intravenous, intramuscular, intraarticular, or subcutaneous injection include, for example, solution, suspension, or emulsion. Methods of topical treatment include, for example, lotions, creams, gels, pastes, ointments, transdermal patches, powders, foams, tinctures, oils, or sprays.

In one embodiment, the subject compositions are formulated as an orally-consumable product, such as, for example a food item, feed item, capsule, pill, or drinkable liquid. An orally deliverable pharmaceutical is any physiologically active substance delivered via initial absorption in the gastrointestinal tract or into the mucus membranes of the mouth. The topic compositions can also be formulated as a solution that can be administered via, for example, injection, which includes intravenously, intraperitoneally, intramuscularly, intrathecally, or subcutaneously. In other embodiments, the subject compositions are formulated to be administered via the skin through a patch or directly onto the skin for local or systemic effects. The compositions can be administered sublingually, buccally, rectally, or vaginally. Furthermore, the compositions can be sprayed into the nose for absorption through the nasal membrane, nebulized, inhaled via the mouth or nose, or administered in the eye or ear.

Orally consumable products according to the invention are any preparations or compositions suitable for consumption, for nutrition, for oral hygiene, or for pleasure, and are products intended to be introduced into the human or animal oral cavity, to remain there for a certain period of time, and then either be swallowed (e.g., food ready for consumption or pills) or to be removed from the oral cavity again (e.g., chewing gums or products of oral hygiene or medical mouth washes). While an orally-deliverable pharmaceutical can be formulated into an orally consumable product, and an orally consumable product can comprise an orally deliverable pharmaceutical, the two terms are not meant to be used interchangeably herein.

Orally consumable products include all substances or products intended to be ingested by humans or animals in a processed, semi-processed, or unprocessed state. This also includes substances that are added to orally consumable products (particularly food and pharmaceutical products) during their production, treatment, or processing and intended to be introduced into the human or animal oral cavity.

Orally consumable products can also include substances intended to be swallowed by humans or animals and then digested in an unmodified, prepared, or processed state; the orally consumable products according to the invention therefore also include casings, coatings, or other encapsulations that are intended to be swallowed together with the product or for which swallowing is to be anticipated.

In one embodiment, the orally consumable product is a capsule, pill, syrup, emulsion, or liquid suspension containing a desired orally deliverable substance. In one embodiment, the orally consumable product can comprise an orally deliverable substance in powder form, which can be mixed with water or another liquid to produce a drinkable orally-consumable product.

Carriers and/or excipients according the subject invention can include any and all solvents, diluents, buffers (such as, e.g., neutral buffered saline, phosphate buffered saline, or optionally Tris-HCl, acetate or phosphate buffers), oil-in-water or water-in-oil emulsions, aqueous compositions with or without inclusion of organic co-solvents suitable for, e.g., IV use, solubilizers (e.g., Polysorbate 65, Polysorbate 80), colloids, dispersion media, vehicles, fillers, chelating agents (e.g., EDTA or glutathione), amino acids (e.g., glycine), proteins, disintegrants, binders, lubricants, wetting agents, emulsifiers, sweeteners, colorants, flavorings, aromatizers, thickeners (e.g. carbomer, gelatin, or sodium alginate), coatings, preservatives (e.g., Thimerosal, benzyl alcohol, polyquaterium), antioxidants (e.g., ascorbic acid, sodium metabisulfite), tonicity controlling agents, absorption delaying agents, adjuvants, bulking agents (e.g., lactose, mannitol) and the like. The use of carriers and/or excipients in the field of drugs and supplements is well known. Except for any conventional media or agent that is incompatible with the target health-promoting substance or with the adjuvant composition, carrier or excipient use in the subject compositions may be contemplated.

In one embodiment, the composition can be made into aerosol formulations so that, for example, it can be nebulized or inhaled. Suitable pharmaceutical formulations for administration in the form of aerosols or sprays are, for example, powders, particles, solutions, suspensions or emulsions. Formulations for oral or nasal aerosol or inhalation administration may also be formulated with carriers, including, for example, saline, polyethylene glycol or glycols, DPPC, methylcellulose, or in mixture with powdered dispersing agents or fluorocarbons. Aerosol formulations can be placed into pressurized propellants, such as dichlorodifluoromethane, propane, nitrogen, fluorocarbons, and/or other solubilizing or dispersing agents known in the art. Illustratively, delivery may be by use of a single-use delivery device, a mist nebulizer, a breath-activated powder inhaler, an aerosol metered-dose inhaler (MDI), or any other of the numerous nebulizer delivery devices available in the art. Additionally, mist tents or direct administration through endotracheal tubes may also be used.

In one embodiment, the composition can be formulated for administration via injection, for example, as a solution or suspension. The solution or suspension can comprise suitable non-toxic, parenterally-acceptable diluents or solvents, such as mannitol, 1,3-butanediol, water, Ringer's solution, or isotonic sodium chloride solution, or suitable dispersing or wetting and suspending agents, such as sterile, non-irritant, fixed oils, including synthetic mono- or diglycerides, and fatty acids, including oleic acid. One illustrative example of a carrier for intravenous use includes a mixture of 10% USP ethanol, 40% USP propylene glycol or polyethylene glycol 600 and the balance USP Water for Injection (WFI). Other illustrative carriers for intravenous use include 10% USP ethanol and USP WFI; 0.01-0.1% triethanolamine in USP WFI; or 0.01-0.2% dipalmitoyl diphosphatidylcholine in USP WFI; and 1-10% squalene or parenteral vegetable oil-in-water emulsion. Water or saline solutions and aqueous dextrose and glycerol solutions may be preferably employed as carriers, particularly for injectable solutions. Illustrative examples of carriers for subcutaneous or intramuscular use include phosphate buffered saline (PBS) solution, 5% dextrose in WFI and 0.01-0.1% triethanolamine in 5% dextrose or 0.9% sodium chloride in USP WFI, or a 1 to 2 or 1 to 4 mixture of 10% USP ethanol, 40% propylene glycol and the balance an acceptable isotonic solution such as 5% dextrose or 0.9% sodium chloride; or 0.01-0.2% dipalmitoyl diphosphatidylcholine in USP WFI and 1 to 10% squalene or parenteral vegetable oil-in-water emulsions.

In one embodiment, the composition can be formulated for administration via topical application onto the skin, for example, as topical compositions, which include rinse, spray, or drop, lotion, gel, ointment, cream, foam, powder, solid, sponge, tape, vapor, paste, tincture, or using a transdermal patch. Suitable formulations of topical applications can comprise in addition to any of the pharmaceutically active carriers, for example, emollients such as carnauba wax, cetyl alcohol, cetyl ester wax, emulsifying wax, hydrous lanolin, lanolin, lanolin alcohols, microcrystalline wax, paraffin, petrolatum, polyethylene glycol, stearic acid, stearyl alcohol, white beeswax, or yellow beeswax. Additionally, the compositions may contain humectants such as glycerin, propylene glycol, polyethylene glycol, sorbitol solution, and 1,2,6 hexanetriol or permeation enhancers such as ethanol, isopropyl alcohol, or oleic acid.

Methods of Treating or Inhibiting Developmental Bone Diseases and/or Other Ailments

The subject invention further provides a method of treating, for example, developmental bone diseases, bone bruising, cartilage damage, bone fracture, or post-infection osteomyelitis bone damage in a subject in need thereof with the novel composition of the subject invention or the two distinct compositions. In certain embodiments, the subject method can also be used to treat or inhibit, either directly or indirectly, the progress of developmental bone diseases.

The subject composition can be administered orally in order to achieve the therapeutic effects. The therapeutic effects can be, for example, systemic, local, tissue-specific, etc. depending on the specific needs of a given application of the invention. In certain embodiments, the subject composition can be administered intranasally, intravenously, intraperitoneally, intramuscularly, intrathecally, intracerebroventricularly, or subcutaneously to a subject.

In certain embodiments, a dosage of about 5 g/day to about 500 g/day, about 10 g/day to about 100 g/day, or about 50 g/day of the marine mineral complex can be administered to the subject. In certain embodiments, the subject receives about 1 g to about 500 g, about 1 g to about 100 g, or about 1 g to about 20 g per hundred pounds of weight of the subject, with the weight measured directly or estimated by girth measurements. In certain embodiments, a dosage of about 1 g/day to about 5000 g/day, about 5 g/day to about 1000 g/day, or about 50 g/day of the strontium salt can be administered to the subject. In certain embodiments, a composition comprising a strontium salt is administered to a subject at least 1 min, 5 min, 10 min, 15 min, 30 min, 1 h, 2 h, 3 h, 4 h, 5 h, or longer either before or after a composition comprising an algae extract is administered to a subject. In certain embodiments, the compositions can be mixed into feed, placed on feed, or directly administered to the subject (e.g., oral administration).

In certain embodiments, the administration of the subject compositions can occur before or after symptoms of a developmental bone disease or related ailment occur. In certain embodiments, the administration of the subject compositions can occur after genetic testing for the presence of genetic markers linked to the developmental bone diseases.

In certain embodiments, the subject methods can resolve sesamoiditis or a break in the sesamoid and, optionally, accompanying desmitis in a subject. In certain embodiments, the subject methods can resolve stifle medial femoral condyle issues, including either small, shallow lucencies or full-sized typical cysts. In certain embodiments, the subject methods can resolve lateral trochlear ridge OCD lesions, medial trochlear ridge lesions, or mid-sagittal ridge lesions in a subject. In certain embodiments, the subject methods can resolve cystic lesions throughout the subject, including, for example, in the lower limb, at distal metacarpal III (MCIII), at a distal PI, and at the distal metatarsal III (MTIII).

### Materials and Methods

Most predominant issues in the investigated horses were OCD defects and cysts, and “sesamoiditis”. “Sesamoiditis” was defined as any significant vascular change in a sesamoid, as demonstrated on radiographs and/or ultrasound. Other areas of concern were navicular disease (as demonstrated on radiographs), OCD fragments, and carpal bone sclerosis. Due to the demographics and relative significance of these issues most horses were yearling to two-year-old thoroughbreds.

The field trial started in various Phases. Phase I was a Control Study in which horses received the marine mineral complex (Aquamin F) and no strontium salt for one month. Phase II comprised selected horses with issues of concern being given a mixture of both a strontium salt (strontium ranelate) and the marine mineral mixture complex (Aquamin F). This phase would carry on for multiple months and eventually blend into a Phase III, which comprises administering the marine mineral complex and strontium salt independently. Between Phase II and Phase III would be a Reassessment Phase in which blood analysis trials would indicate if other strontium salts were at least a serum equivalent to the strontium ranelate (formulation issues) and if dosing regimen was effective.

### Phase I

Twenty-nine horses, most all young thoroughbreds, were included in Phase I. Of these six horses had 6 OCD cysts; seven horses had 9 OCD defects; 14 horses had 27 sesamoid issues; three horses had 5 sclerosis issues; and one horse had 1 OCD fragment issue. Based on radiographic evaluation, none had any change in radiographic evaluation after approximately 30 days.

### Phase II

Phase II comprises the administration of the subject composition, comprising strontium ranelate and the marine algae mineral complex marketed under the name “Aquamin®”. Dosage of the marine mineral complex is about 50 g/day. Horses would receive approximately 1 gram per hundred pounds with weight estimated by girth measurements, and receive a 32-day supply (i.e., a bucket) at a time. The population of horses is young Thoroughbreds with the main ailments being sesamoiditis (increased vascularity of any degree), OCD lesions at various locations, OCD cysts at various locations, navicular radiographic changes, third carpal bone sclerosis, sesamoid fractures, mid-sagittal ridge at distal metacarpal III (MCIII) lucencies, and one horse had an OCD fragment that was followed radiographically. All evaluations were done at the radiographic level with multiple views if possible; ultrasound of sesamoid abaxial borders was used in a few cases of sesamoiditis were suspensory desmitis was suspected. Resolution grades, primarily based on were established at Zero, mild (1-30%), moderate (31-60%), significant (61-94%), and full (95-100%).

In certain embodiments, the subject compositions can be used in methods to treat a subject suffering from osteoarthritis. To obtain the maximum efficacy, the treatment should be initiated in subjects with early stages of osteoarthritis pathogenesis, or at least as early as possible.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### EXAMPLES

### Example 1-Treatment of Developmental Bone Diseases

Sixty-one horses participated in Phase II. 54 were young yearlings to turning two Thoroughbreds with primary issues of OCD defects or cysts, and sesamoiditis. Five horses had primary issues of navicular disease with one, an older thoroughbred, with carpal sclerosis. One older quarter horse had a slight lucency at the distal medial femoral condyle of the femur. A total of 184 buckets (i.e., 32-day supply) were dispensed in total with each horse averaging using 3 buckets so the average horse was on the supplement 3 months, some as short as one month, others as long as 6 months. Not one horse showed any adverse effects from being on the supplement.

Thirty-five horses presented with various level of radiographic “sesamoiditis” with a total of 72 sesamoids surveyed. 43 sesamoids showed zero resolution, 9 showed mild, 17 showed moderate, and 3 significant. There were no full resolutions. Of interest was that all three of the significant resolutions had their supplement orally dosed versus adding as a top dressing to the feed. 5 of the zero resolution sesamoids also were diagnosed using ultrasound with accompanying desmitis and in which case the vascular change in the sesamoid worsened. In two cases, the break in the sesamoid seen on ultrasound disappeared with treatment. It was determined that exercise with desmitis was detrimental to the sesamoid's level of radiographic “sesamoiditis”, and stall rest was beneficial. One horse went back to work before the desmitis was resolved and the desmitis and sesamoiditis worsened.

Seven horses, which includes all but one young Thoroughbreds, presented with a total of 9 stifle medial femoral condyle issues, either small shallow lucencies or full-sized typical cysts. There was 7 stifles that showed zero resolution, one cyst that showed significant resolution, and one small area in a yearling thoroughbred that had full resolution. Another older Quarter Horse with a small defect showed zero resolution.

Twelve horses, all young thoroughbreds, presented with a total of 16 lateral trochlear ridge OCD lesions and one horse, a young thoroughbred, presented with 2 medial trochlear ridge lesions. Of the 18 lesions, 4 showed zero response (All LTR's), 2 showed mild resolution, 6 showed moderate resolution, and 5 showed significant resolution, and one, a yearling with a small MFC defect, showed full resolution. Four horses had significant joint effusion (3 lateral trochlear ridges (LTR's), 1 with medial trochlear ridges (MTR's)) and associated moderate lameness. All horses over the course of one month had less joint effusion and became sound (i.e., not lame).

Seven horses had 9 cystic lesions in the lower limb; 7 at distal MCIII, and one at distal PI, and one the distal MTIII. Three horses had zero resolution; one had an atypical appearance; one had a very shallow (about 2 mm in diameter) distal metacarpal III (MCIII) lucency. Two lesions had moderate resolution, and four lesions had significant to full resolution. One horse had clinical lameness associated with a distal MCIII cyst and became sound on the supplement within 30 days.

Miscellaneous cases included two horses that had third carpal bone (CIII) sclerosis, which responded to the subject treatment. Four horses had radiographic evidence of navicular change and these had zero resolution, and two horses had mid-sagittal ridge lesions that had moderate to significant resolution.

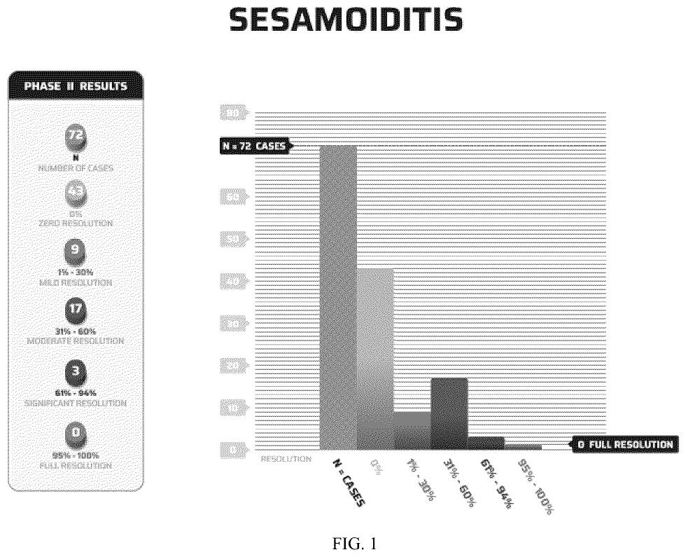

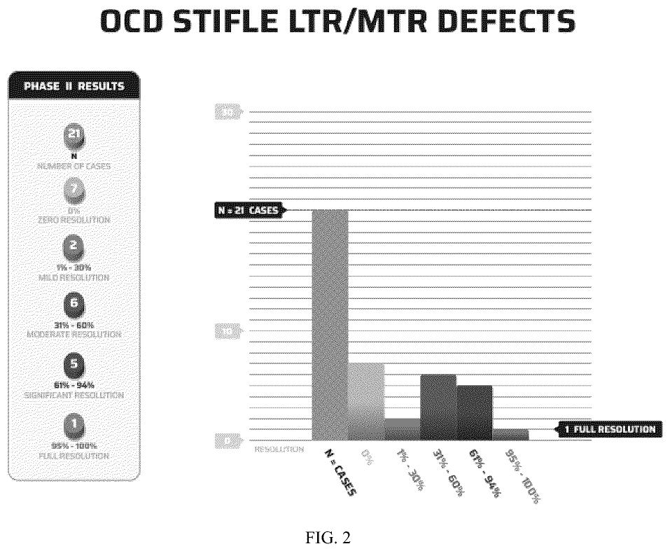

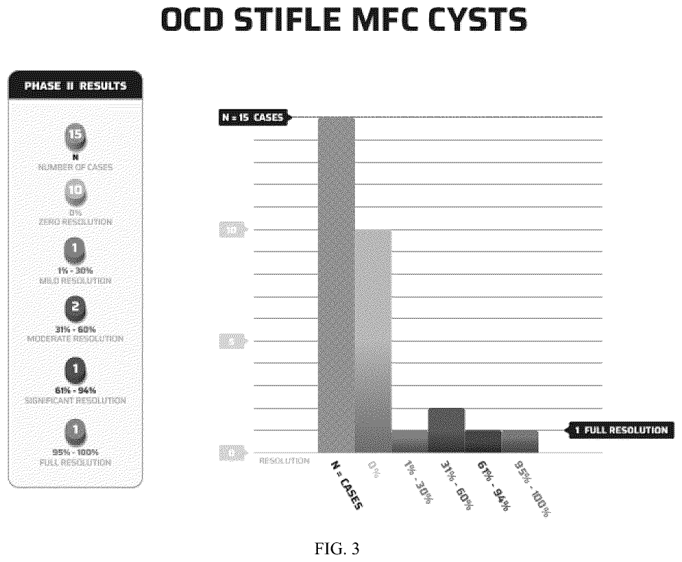

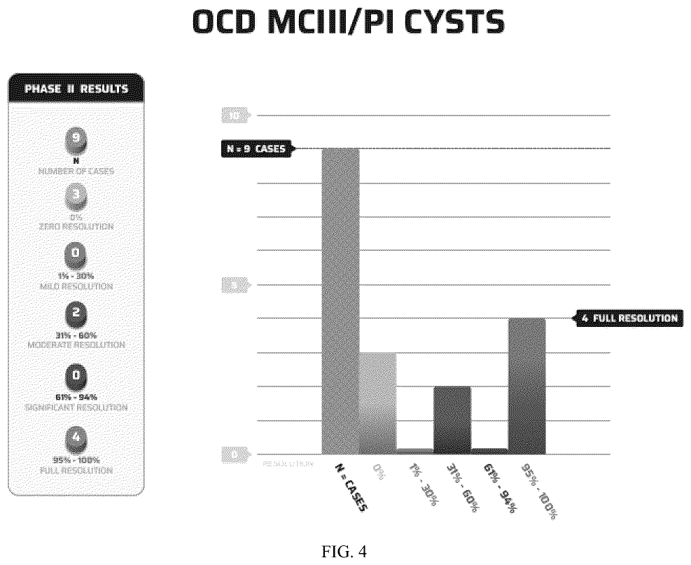

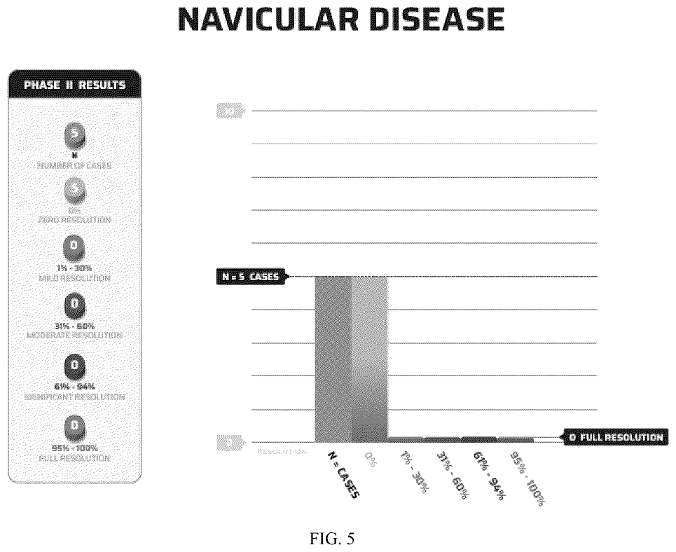

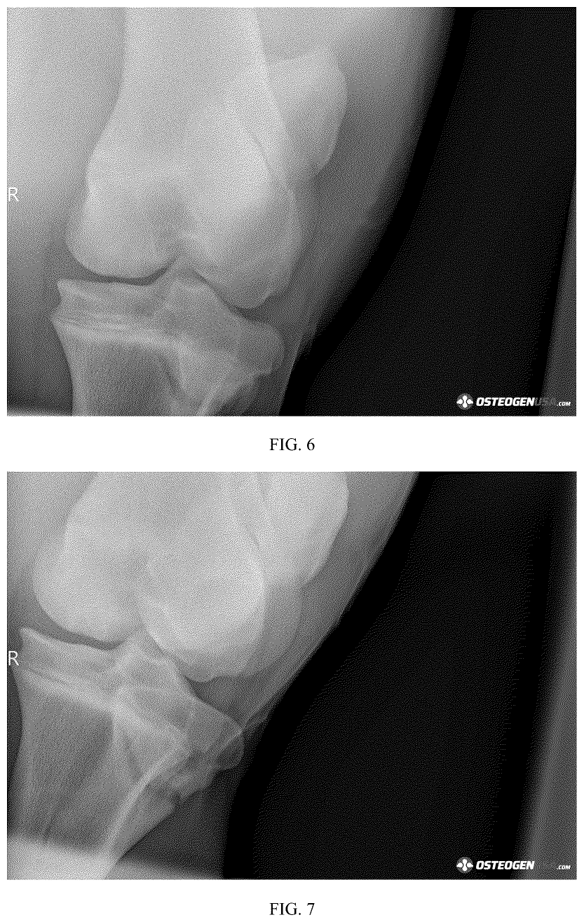

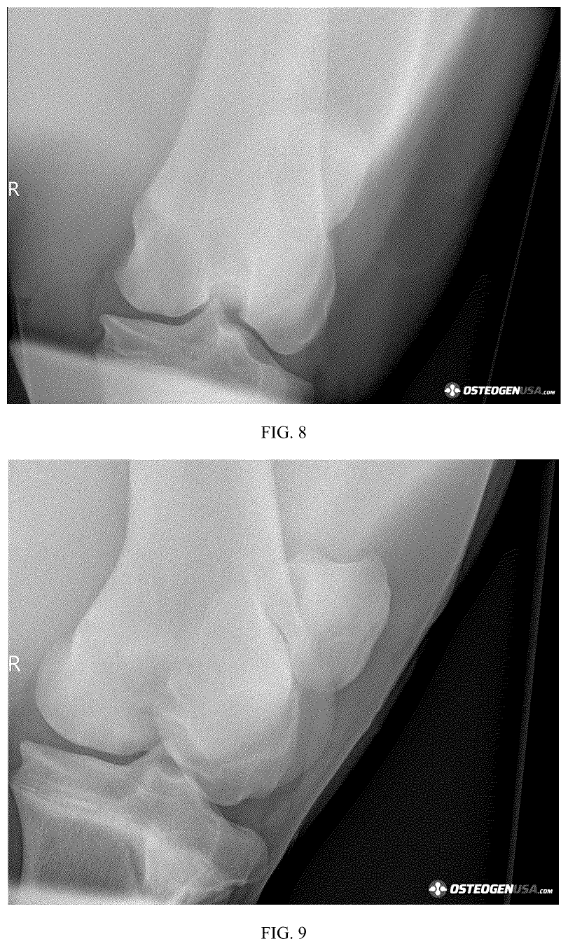

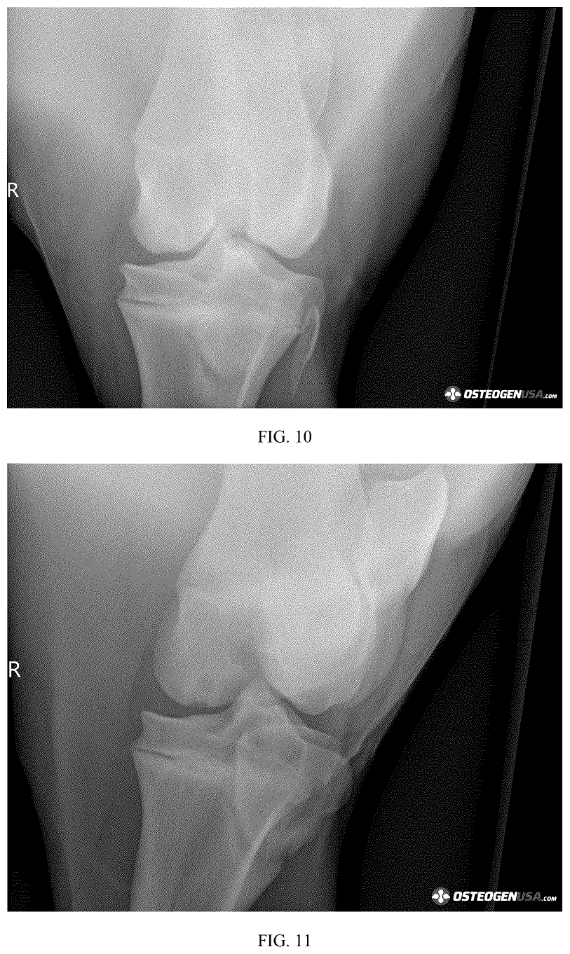

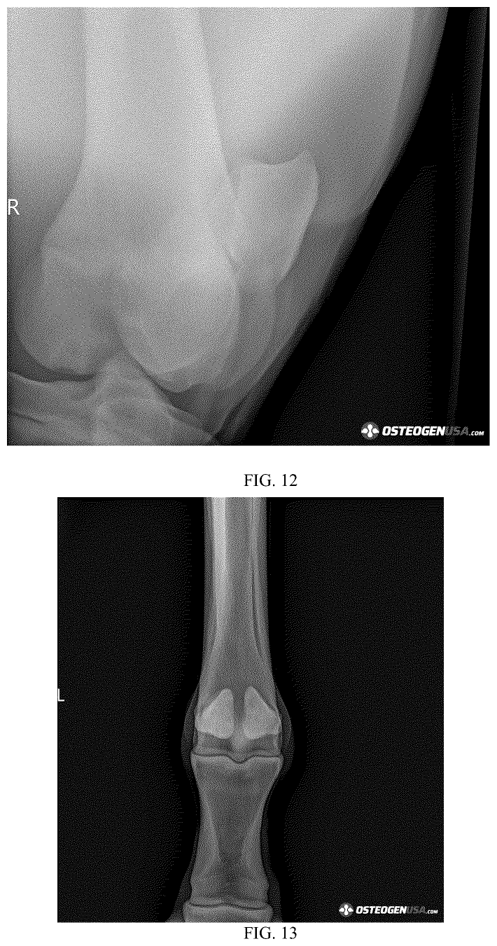

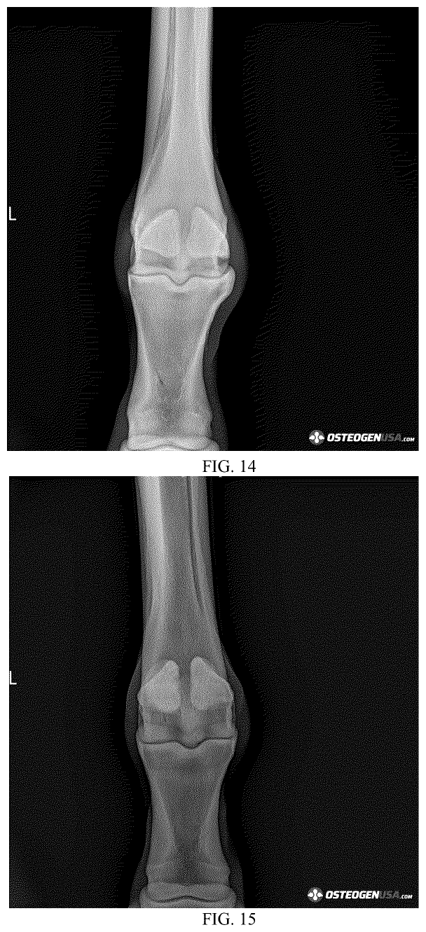

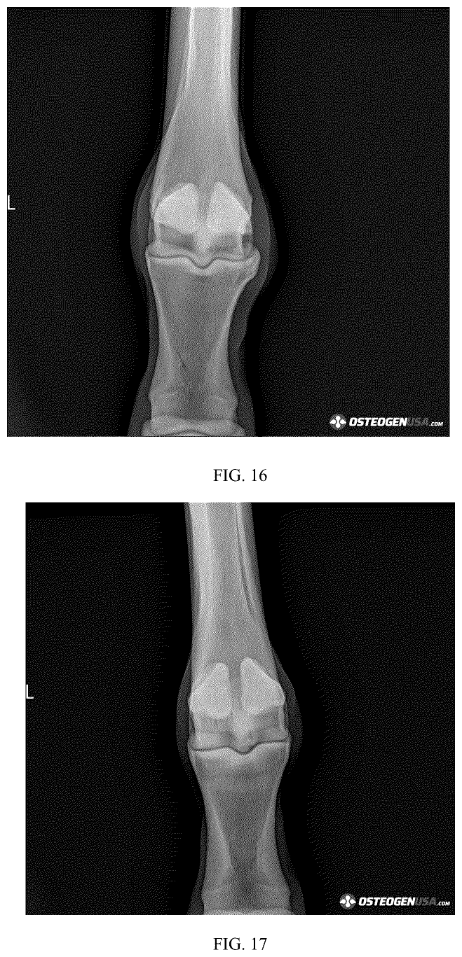

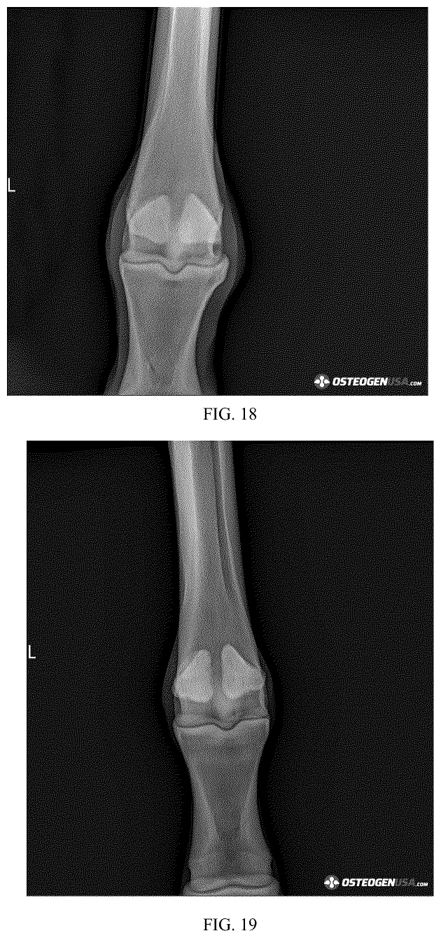

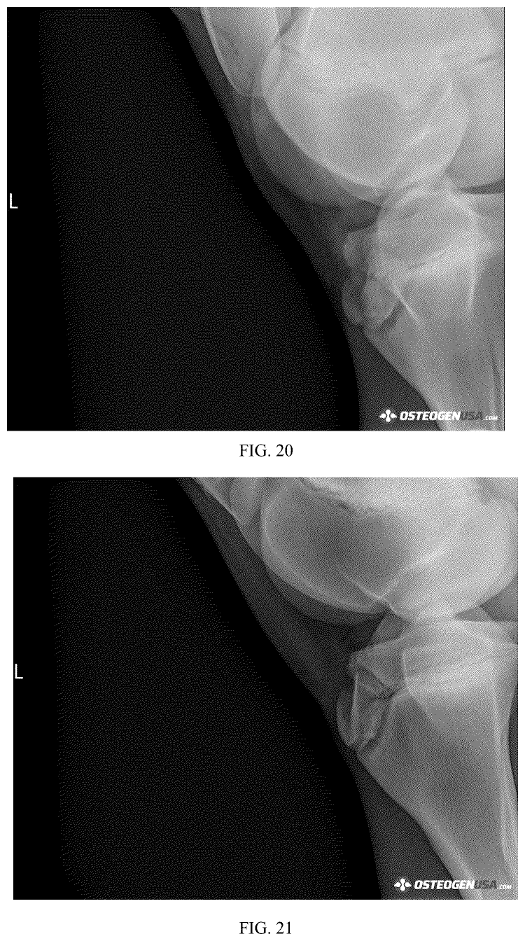

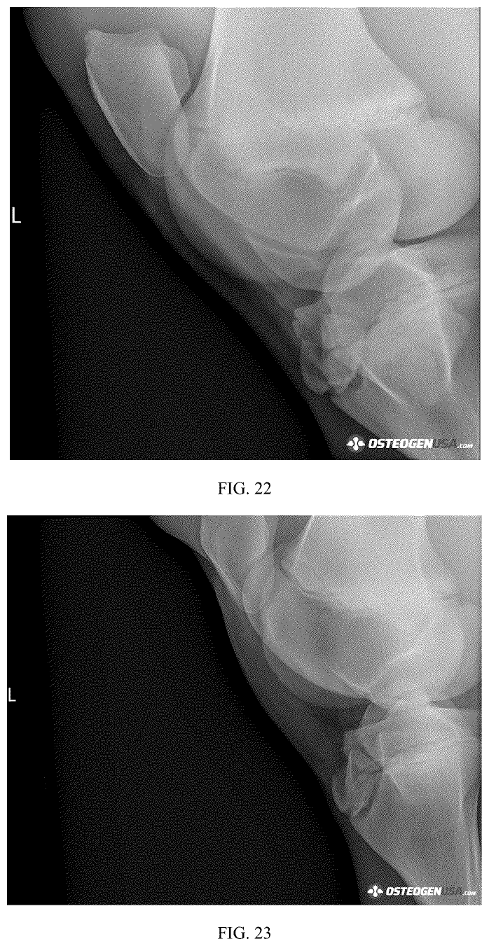

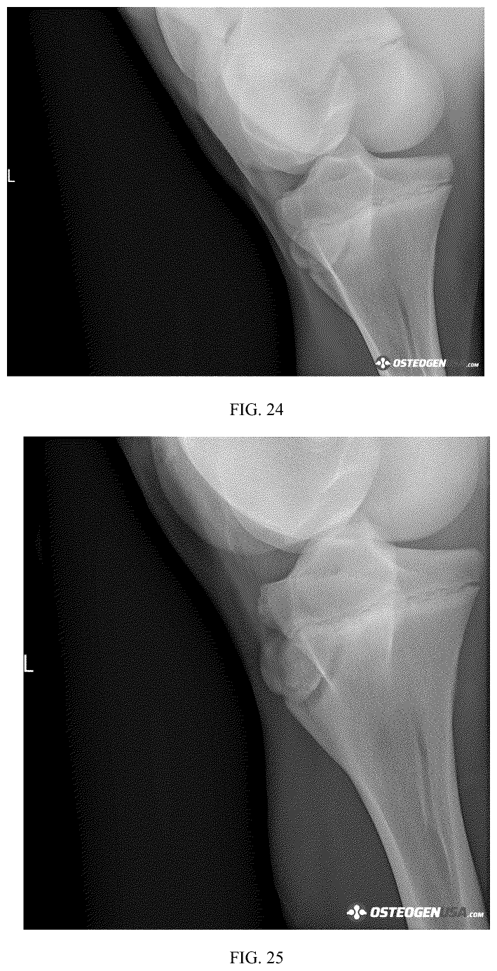

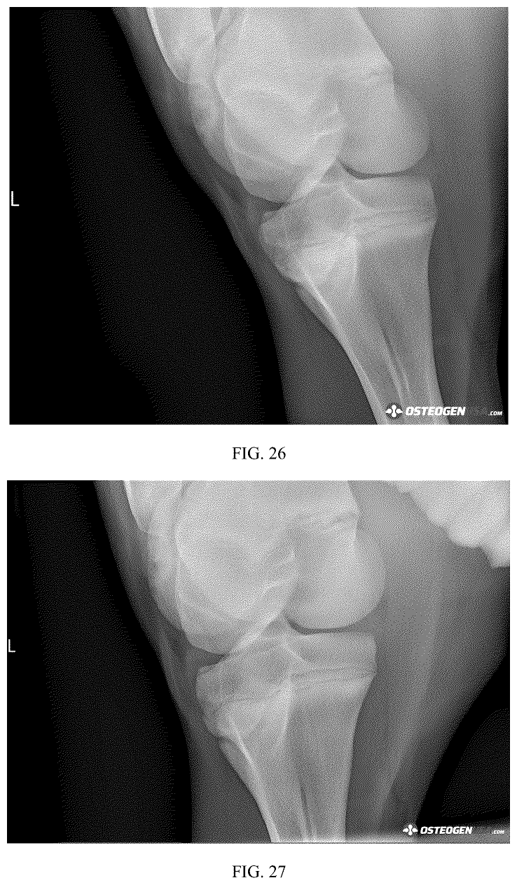

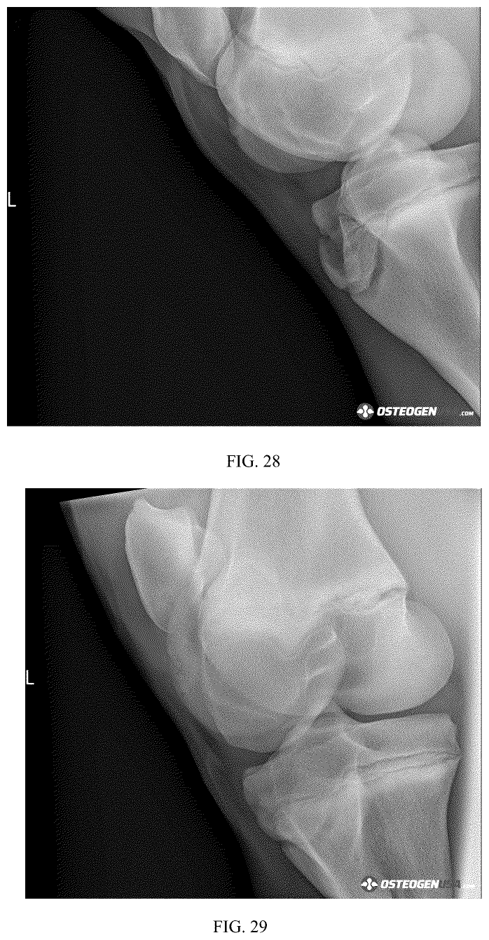

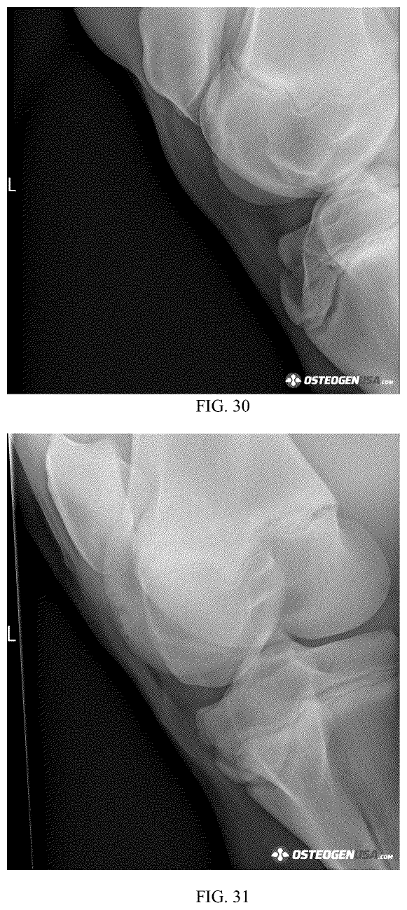

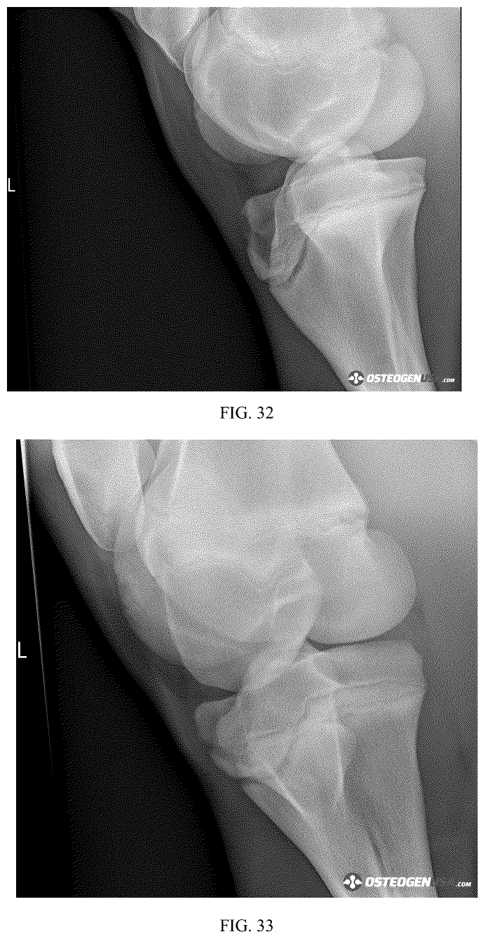

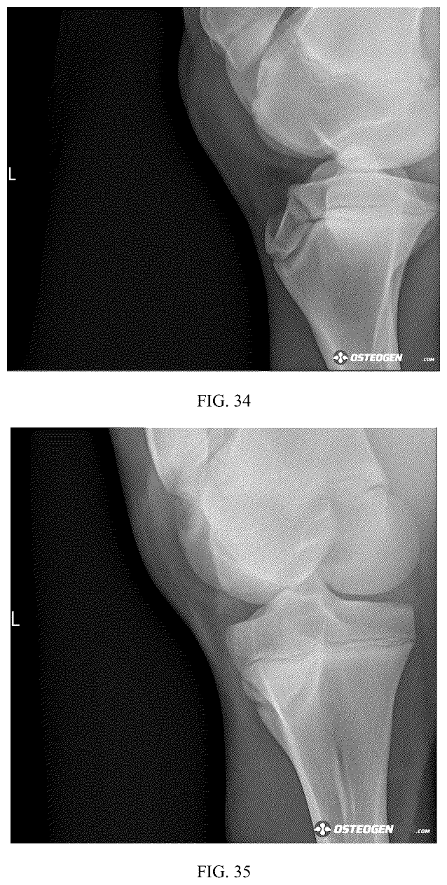

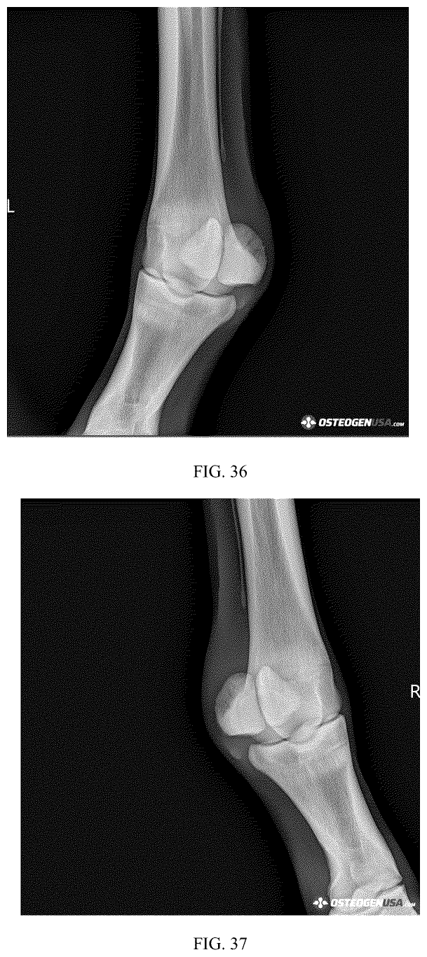

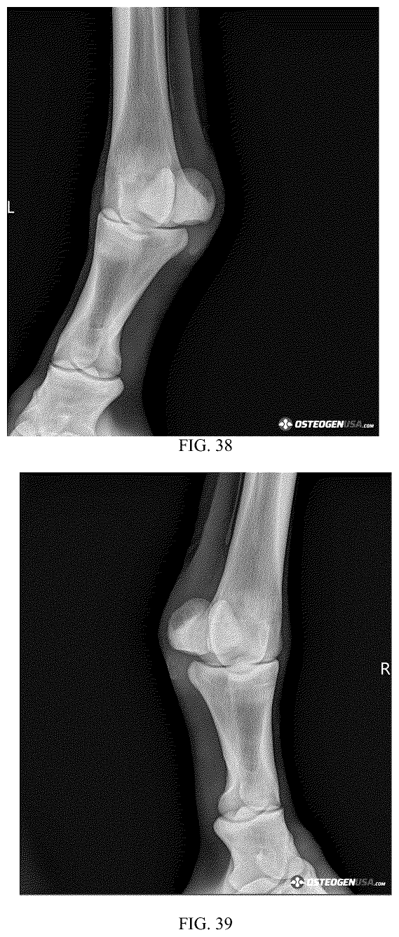

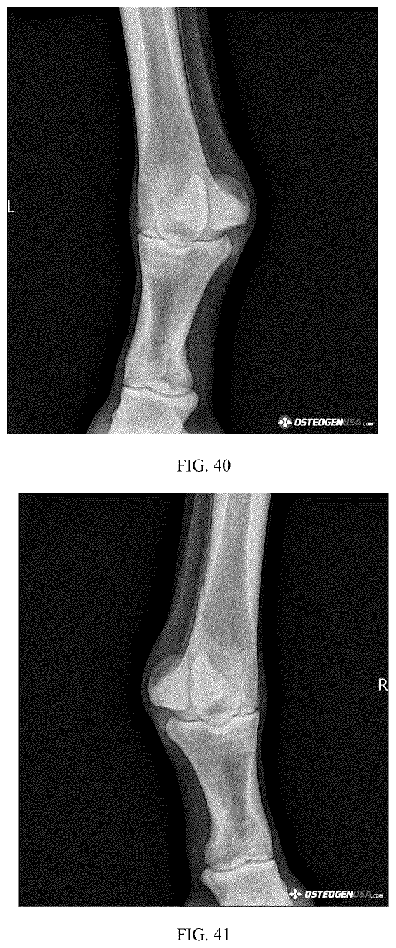

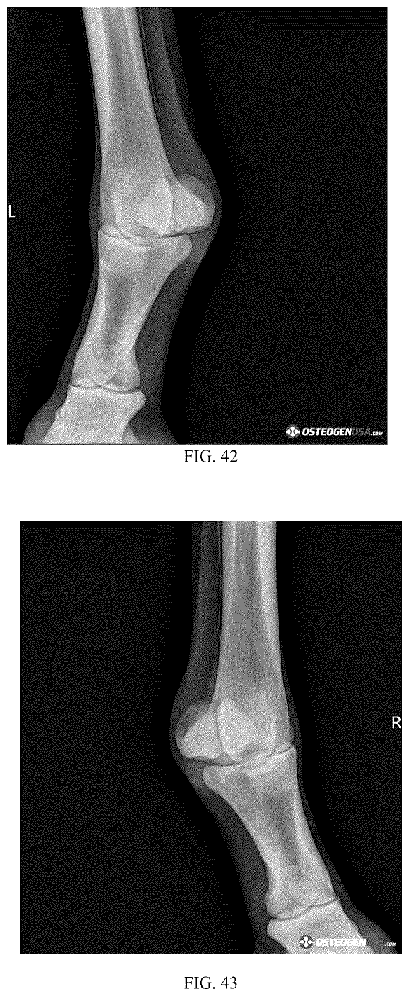

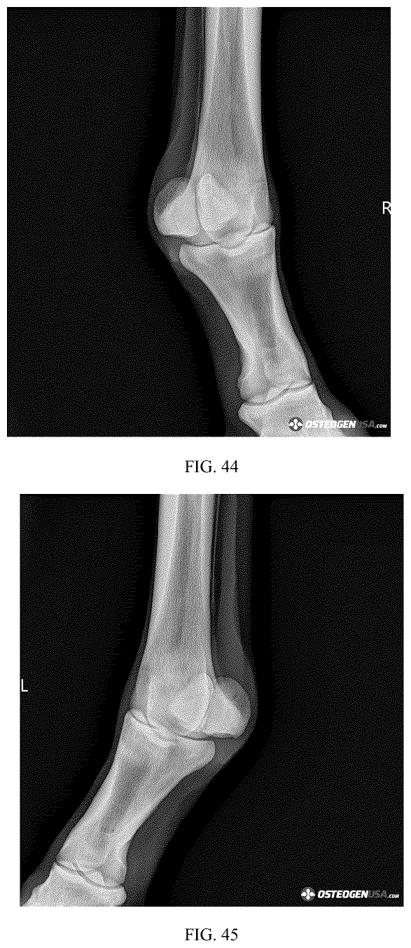

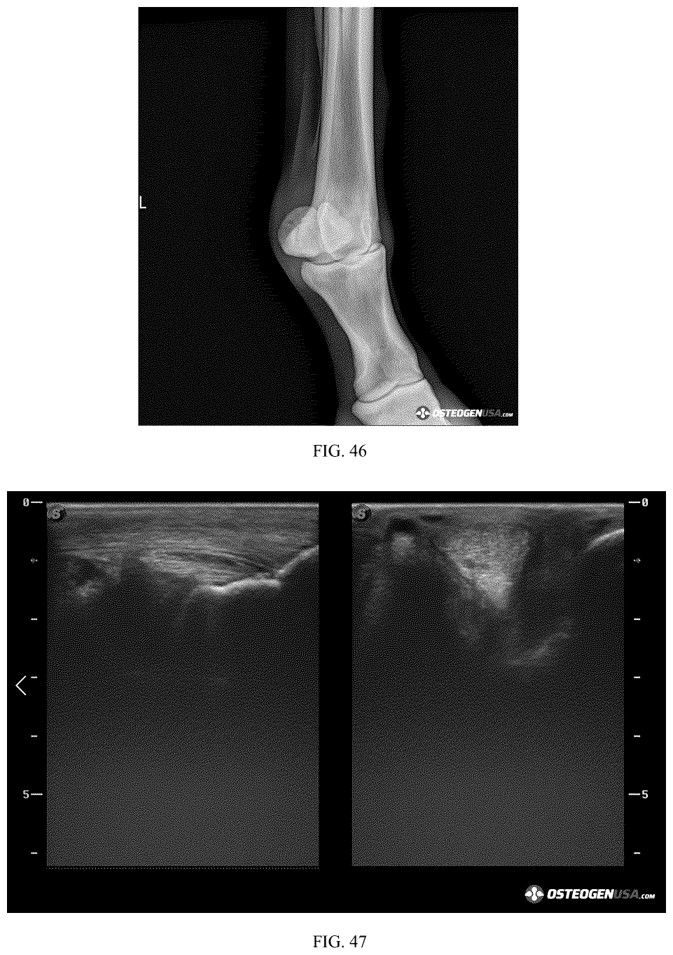

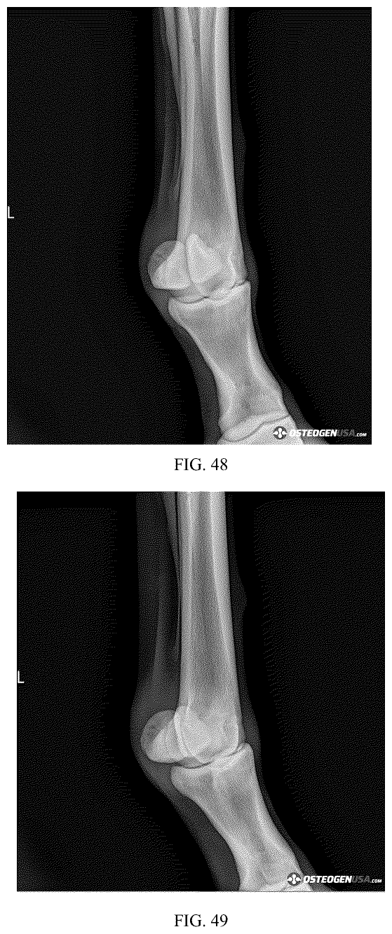

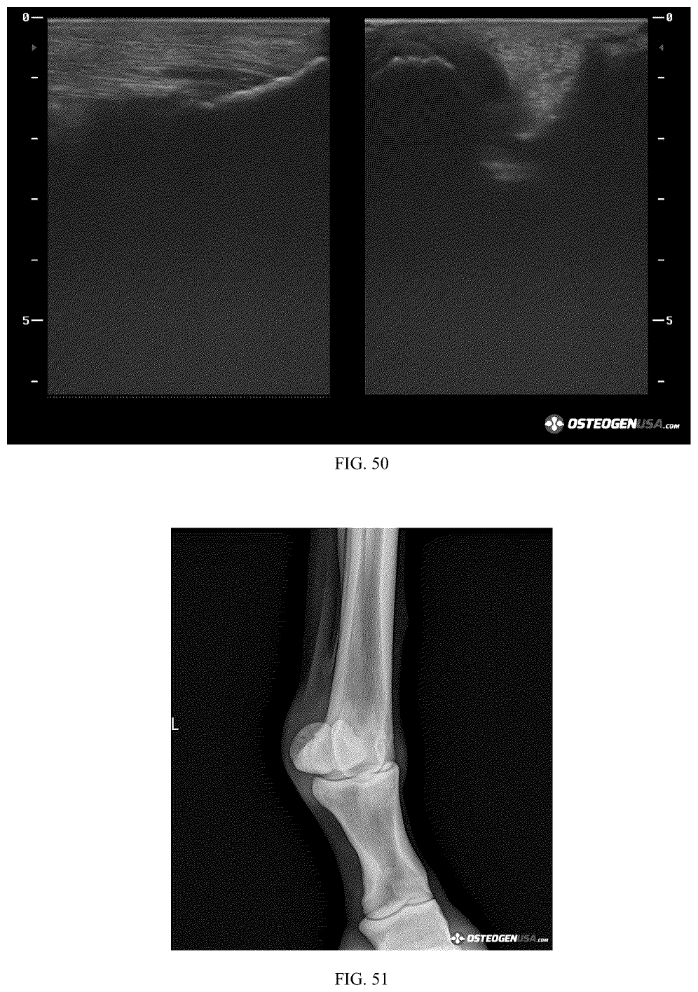

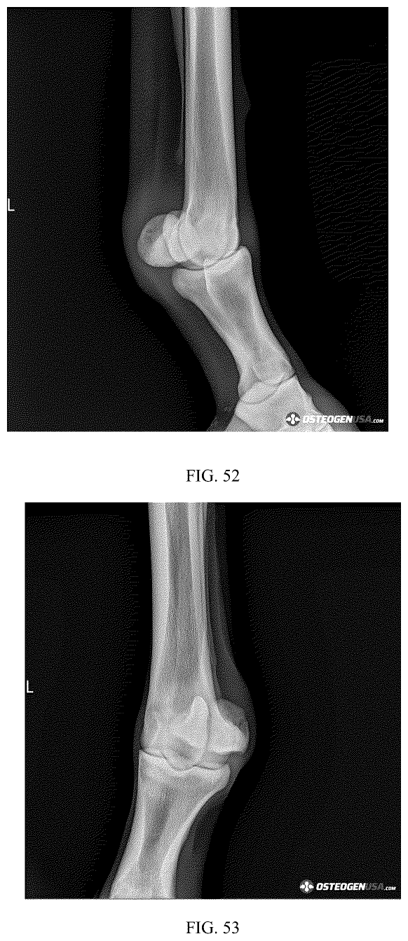

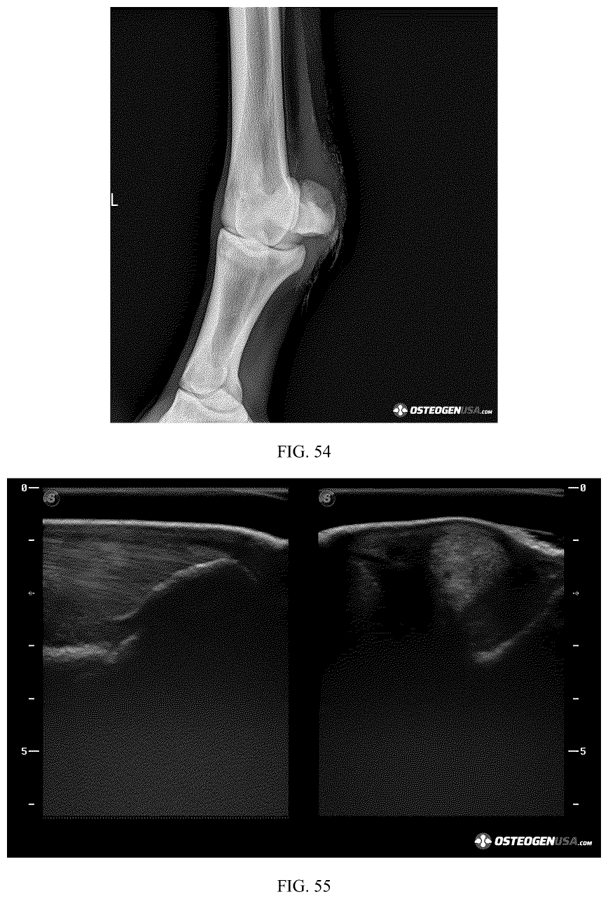

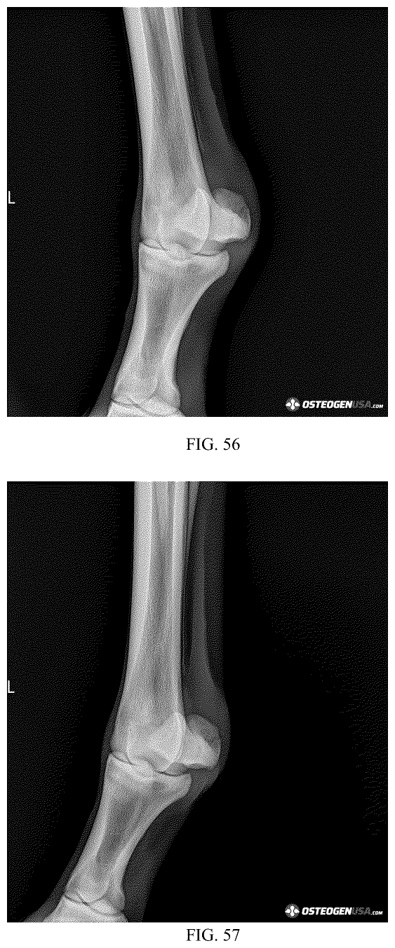

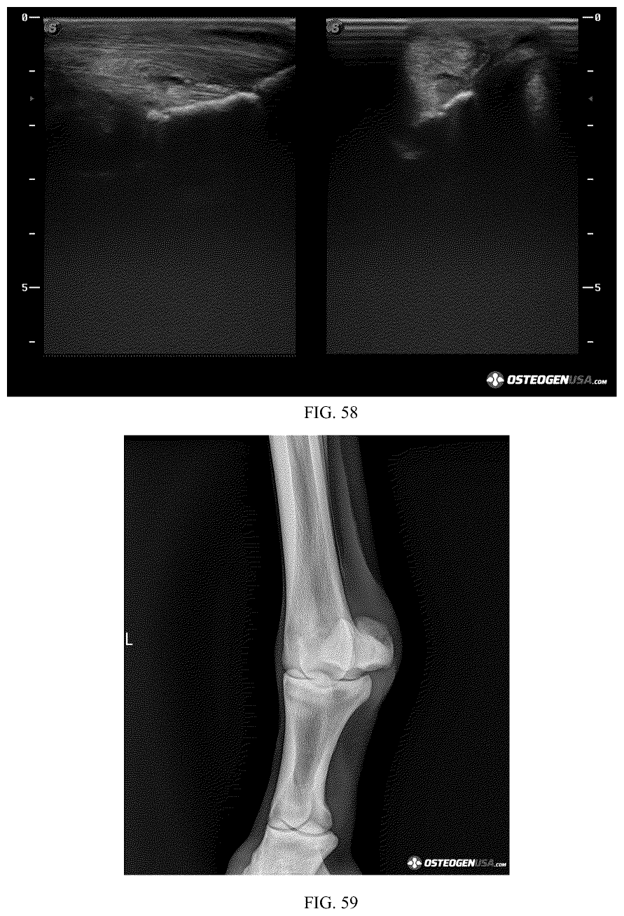

FIGS. 2-4 and 6-35 show the effectiveness and progression of OCD cyst or defect treatment before and during treatment with the subject compositions. FIGS. 1 and 36-59 show the effectiveness and progression of sesamoiditis treatment before and during treatment with the subject compositions. FIG. 5 shows the effectiveness of navicular disease treatment with the subject compositions

With regards to safety, there were no adverse effects of any kind with any recipient. The supplementation of strontium ranelate combined with the algae mineral complex was an effective treatment of OCD bone cysts and lateral trochlear ridge lesions. Some horses with bilateral lesions had one lesion respond much more dramatically than the contralateral lesion. Resolution using the subject compositions was least effective in large stifle cysts, sesamoiditis, and navicular bones. Sesamoiditis had complicating side factors involving associated desmitis and exercise compared to no exercise. Ultrasound evidence of the vascular channels filling in was the most supportive finding during phase II. The resolution of the joint effusions and lameness after receiving the supplement was very interesting. Joint effusion and lameness with these cases is generally associated with cartilage fibrillation and deterioration.

Approximately 4 months into the trial, strontium blood levels were measured in a small group of horses. Calcium levels were not measured for not only financial constraints, but also previous work had demonstrated that supplementation with calcium did not result in an increase in Calcium serum blood levels. (Li et al., 2009). To measure strontium blood levels, Inductively Coupled Plasma Mass Spectrometry was used (Eurofins Lab, (Wisconsin USA)). Two test samples from random study horses indicated 21 and 31 μmol/L strontium levels.

A group of six horses were utilized and were administered the subject compositions for 14 days total. The compositions were added to morning and evening feed as a top dressing. All subjects received 50 g of “Aquamin” daily given as 25 g in morning and 25 g in evening. Three horses received Strontium ranelate at 10 g, 12 g, and 15 g daily split between the morning and evening. Three horses received Strontium citrate at 10 g, 12 g, and 14 g daily split morning and evening. Blood samples were taken a day 0, 1, 5, 8, 12, and 15 days. Bloods were drawn first thing in the morning before any supplements were fed.

Overall, blood levels were quite low compared to human blood levels receiving 2 grams per day with a maximum of about 7.5 μmole/L on two ranelate horses and a maximum of 9.3 and 9.5 μmole/L on two different citrate horses. Human levels were typically measured around 140-150 μmole/L. (AusProtos, 2013).

Strontium citrate levels were comparable with strontium ranelate levels. This is a surprising finding as strontium ranelate had very limited availability and is much more expensive than the strontium citrate.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1. Aslam M N, Kreider J M, Paruchuri T, Bhagavathula N, DaSilva M, Zernicke R F, Goldstein S A, Varani J. A mineral-rich extract from the red marine algae Lithothamnion calcareum preserves bone structure and function in female mice on a Western-style diet. Calcif Tissue Int. 2010 April; 86(4):313-24.
2. Australian Public Assessment Report for strontium ranelate. Therapeutic Goods Administration. 2013 January: 1-48.
3. Cardenas D A. Elemental Mapping Of Bone Strontium In Rats Treated With Strontium Ranelate And Strontium Citrate Using 2D Micro-XRF And 3D Dual Energy K-edge Subtraction X-ray Imaging. Toronto Metropolitan University. Thesis.
4. Frestedt J L, Kuskowski M A, Zenk, J. A natural seaweed derived mineral supplement (Aquamin F) for knee osteoarthritis: A randomised, placebo controlled pilot study. Nutr J. 2009; 8(7).
5. Frestedt J L, Walsh M, Kuskowski M A, Zenk J L. A natural mineral supplement provides relief from knee osteoarthritis symptoms: a randomized controlled pilot trial. Nutr J. 2008; February 17: 7-9.
6. Li Z, Lu W W, Chiu P K, Lam R W, Xu B, Cheung K M, Leong J C, Luk K D. Strontium-calcium coadministration stimulates bone matrix osteogenic factor expression and new bone formation in a large animal model. J Orthop Res. 2009 June; 27(6):758-62.
7. Marx D, Rahimnejad Yazdi A, Papini M, Towler M. A review of the latest insights into the mechanism of action of strontium in bone. Bone Rep. 2020 Apr. 24; 12:100273.
8. Nielsen B D, Cate R E, O'Connor-Robison C I. A Marine Mineral Supplement Alters Markers of Bone Metabolism in Yearling Arabians. J. Equine Vet. Sci. 2010; 30(8): 419-424.
9. O'Gorman D M, Tierney C M, Brennan O, O'Brien F J. The marine-derived, multi-mineral formula, Aquamin, enhances mineralisation of osteoblast cells in vitro. Phytother Res. 2012 March; 26(3):375-80.
10. Reginster J Y, Beaudart C, Neuprez A, Bruyère O. Strontium ranelate in the treatment of knee osteoarthritis: new insights and emerging clinical evidence. Ther Adv Musculoskelet Dis. 2013 October; 5(5):268-76.
11. Shorr E, Carter A C. The usefulness of strontium as an adjuvant to calcium in the remineralization of the skeleton in man. Bull Hosp Joint Dis. 1952 April; 13(1):59-66.
12. Wohl G R, Chettle D R, Pejović-Milić A, Druchok C, Webber C E, Adachi J D, Beattie K A. Accumulation of bone strontium measured by in vivo XRF in rats supplemented with strontium citrate and strontium ranelate. Bone. 2013 January; 52(1):63-9.
13. Mirza F S, Azim S, Bhargava A. Change in Bone Mineral Density with Strontium Citrate: An Illusion or Reality. J Nutrition Health Food Sci. 2016 September; 4(3):1-3.
14. Marie P J, Garba M T, Hott M, Miravet L. Effect of low doses of stable strontium on bone metabolism in rats. Miner Electrolyte Metab. 1985; 11(1):5-13.
15. Marie P J, Hott M. Short-term effects of fluoride and strontium on bone formation and resorption in the mouse. Metabolism. 1986 June; 35(6):547-51.
16. Grynpas M D, Marie P J. Effects of low doses of strontium on bone quality and quantity in rats. Bone. 1990; 11(5):313-9.
17. Arlot M E, Jiang Y, Genant H K, Zhao J, Burt-Pichat B, Roux J P, Delmas P D, Meunier P J. Histomorphometric and microCT analysis of bone biopsies from postmenopausal osteoporotic women treated with strontium ranelate. J Bone Miner Res. 2008 February; 23(2):215-22.
18. Meunier P J, Roux C, Seeman E, Ortolani S, Badurski J E, Spector T D, Cannata J, Balogh A, Lemmel E M, Pors-Nielsen S, Rizzoli R, Genant H K, Reginster J Y. The effects of strontium ranelate on the risk of vertebral fracture in women with postmenopausal osteoporosis. N Engl J Med. 2004 Jan. 29; 350(5):459-68.
19. Reginster J Y, Seeman E, De Vernejoul M C, Adami S, Compston J, Phenekos C, Devogelaer J P, Curiel M D, Sawicki A, Goemaere S, Sorensen O H, Felsenberg D, Meunier P J. Strontium ranelate reduces the risk of nonvertebral fractures in postmenopausal women with osteoporosis: Treatment of Peripheral Osteoporosis (TROPOS) study. J Clin Endocrinol Metab. 2005 May; 90(5):2816-22.
20. Bruyere O, Delferriere D, Roux C, Wark J D, Spector T, Devogelaer J P, Brixen K, Adami S, Fechtenbaum J, Kolta S, Reginster J Y. Effects of strontium ranelate on spinal osteoarthritis progression. Ann Rheum Dis. 2008 March; 67(3):335-9.
21. Gulhan I, Bilgili S, Gunaydin R, Gulhan S, Posaci C. The effect of strontium ranelate on serum insulin like growth factor-1 and leptin levels in osteoporotic post-menopausal women: a prospective study. Arch Gynecol Obstet. 2008 November; 278(5):437-41.
22. Namazi H. Efficacy and safety of oral strontium ranelate for the treatment of knee osteoarthritis: rationale and design of randomised, double-blind, placebo-controlled trial. Curr Med Res Opin. 2012 April; 28(4):609; author reply 609-10.
23. Tat S K, Pelletier J P, Mineau F, Caron J, Martel-Pelletier J. Strontium ranelate inhibits key factors affecting bone remodeling in human osteoarthritic subchondral bone osteoblasts. Bone. 2011 September; 49(3):559-67.
24. Alexandersen P, Karsdal M A, Qvist P, Reginster J Y, Christiansen C. Strontium ranelate reduces the urinary level of cartilage degradation biomarker CTX-II in postmenopausal women. Bone. 2007 January; 40(1):218-22.
25. Alexandersen P, Karsdal M A, Byrjalsen I, Christiansen C. Strontium ranelate effect in postmenopausal women with different clinical levels of osteoarthritis. Climacteric. 2011 April; 14(2):236-43.

26. Li Z, Lu W W, Chiu P K, Lam R W, Xu B, Cheung K M, Leong J C, Luk K D. Strontium-calcium coadministration stimulates bone matrix osteogenic factor expression and new bone formation in a large animal model. J Orthop Res. 2009 June; 27(6):758-62.

1. A method of inhibiting the onset of a developmental bone disease or treating a developmental bone disease, bone bruising, cartilage damage, bone fracture, or post-infection osteomyelitis bone damage, the method comprising administering a single composition to the subject comprising an effective amount of an non-viable marine algae culture or an extract thereof and at least one strontium salt; or a first composition to the subject comprising an effective amount of an non-viable marine algae culture or an extract thereof and a second composition comprising at least one strontium salt.

2. The method of claim 1, wherein the developmental bone disease is osteochondrosis (OCD), sesamoiditis, bone sclerosis, osteoarthritis, navicular disease, or any combination thereof.

3. The method of claim 1, wherein the first composition is a solution, a suspension, an emulsion, an inhalable powder, an aerosol, or a spray, the second composition is a solution, a suspension, an emulsion, an inhalable powder, an aerosol, or a spray, and the single composition is a solution, a suspension, an emulsion, an inhalable powder, an aerosol, or a spray.

4. The method of claim 1, wherein the single, first, and/or second composition is administered orally; and further comprises further comprising at least one solvent, at least one additive, or a combination thereof, wherein the solvent is water, ethanol, butanol, propanol, aliphatic hydrocarbons, alicyclic hydrocarbons, xylene, toluene, ketones, isopropyl alcohol, or any combination thereof and the additive is a filler, binder, disintegrant, diluent, glidant, emulsifying agent, suspending agent, stabilizer, enhancer, flavor, dye, pigment, pH adjusting agent, buffer, retarding agent, wetting agent, adhesion-promoting compound, surface active agent, preservatives, antioxidant, defoamer, anti-skinning agent, texturizer, emulsifying agent, lubricant, solubility controlling agent, chelating agent, conventional carrier composition, vitamin, or any combination thereof.

5. The method of claim 1, wherein the non-viable marine algae culture or extract thereof comprises a Lithothamnion sp., calcium carbonate, and magnesium carbonate.

6. The method of claim 5, wherein the Lithothamnion sp. is Lithothamnion calcareum, Lithothamnion corallioides, or Lithothamnion crispatum.

7. The method of claim 1, wherein the marine algae extract comprises about 0.01% to about 99% by weight of the total first composition or the total single composition and the strontium salt comprises about 0.01% to about 99% by weight of the total second composition or the total single composition.

8. The method of claim 1, wherein the strontium salt is strontium ranelate, strontium citrate, strontium phosphate, or any combination thereof.

9. The method of claim 1, wherein the non-viable marine algae extract is administered at a dose of about 1 g/day to about 500 g/day and the strontium salt is administered at a dose of about 1 g/day to about 500 g/day.

10. The method of claim 1, wherein the administration of the first composition occurs about 1 h to about 5 h before or after the administration of the second composition.

11. (canceled)

12. The method of claim 9, wherein the strontium salt is strontium citrate.

13. The method of claim 12, wherein the dose of strontium citrate is about 20 g/day.

14. The method of claim 9, wherein the algae extract is Aquamin F.

15. The method of claim 14, wherein the dose of Aquamin F is about 50 g/day.

16. The method of claim 14, wherein the algae extract is Aquamin F administered at about 50 g/day.

17. A method of treating bone bruising, cartilage damage, bone fracture, or post-infection osteomyelitis bone damage, the method comprising administering a single composition to the subject comprising an effective amount of an non-viable marine algae culture or an extract thereof and at least one strontium salt, wherein the at least one strontium salt is strontium ranelate.

18. The method of claim 17, wherein the single composition is a solution, a suspension, an emulsion, an inhalable powder, an aerosol, or a spray.

19. The method of claim 17, wherein the single composition is administered orally; and wherein the single composition further comprises at least one solvent, at least one additive, or a combination thereof, wherein the solvent is water, ethanol, butanol, propanol, aliphatic hydrocarbons, alicyclic hydrocarbons, xylene, toluene, ketones, isopropyl alcohol, or any combination thereof and the additive is a filler, binder, disintegrant, diluent, glidant, emulsifying agent, suspending agent, stabilizer, enhancer, flavor, dye, pigment, pH adjusting agent, buffer, retarding agent, wetting agent, adhesion-promoting compound, surface active agent, preservatives, antioxidant, defoamer, anti-skinning agent, texturizer, emulsifying agent, lubricant, solubility controlling agent, chelating agent, conventional carrier composition, vitamin, or any combination thereof.

20. The method of claim 17, wherein the at least one strontium salt further comprises strontium citrate, strontium phosphate, or a combination thereof.

21. The method of claim 17, wherein the treating comprises one or a combination of the following:
a) resolving sesamoiditis or a break in the sesamoid and, optionally, accompanying desmitis;
b) resolving stifle medial femoral condyle issues; and
c) resolving bone bruising, cartilage damage, bone fracture, or post-infection osteomyelitis bone damage.
